(19) United States
(12) Patent Application Publication
(10) Pub. No.: US 2009/0061020 A1
(43) Pub. Date: Mar. 5, 2009

(54) Brimonidine Compositions for Treating Erythema

(76) Inventors: Klaus P. Theobald, Paoli, PA (US); Christopher V. Powala, Radnor, PA (US)

Correspondence Address: HOFFMANN & BARON, LLP, 6900 JERICHO TURNPIKE, SYOSSET, NY, 11791, US

(21) Appl. No.: 12/193,098

(22) Filed: Aug. 18, 2008

Related U.S. Application Data
(60) Provisional application No. 60/967,191, filed on Aug. 31, 2007.

(51) Int. Cl. A61K 31/498; A61K 8/29; A61P 17/00; A61Q 19/00

(52) U.S. Cl. 424/617; 514/249

(57) ABSTRACT

The present invention is directed to a pharmaceutical composition including brimonidine tartrate in an amount from about 0.17 percent by weight to about 0.19 percent by weight in a pharmaceutically acceptable carrier such as a gel or cream. The invention also relates to a method of treating erythema in a patient with rosacea by administering the composition of the invention to the site of erythema on the skin of the patient.

### PRIOR ART

Brimonidine tartrate in aqueous solution (0.15% and 0.20%) is a known for ophthalmic use. It is sold by Allergan under the name ALPHAGAN® P.

It has been discovered that brimonidine tartrate is also useful in treating erythema caused by rosacea. Creams and gels containing brimonidine tartrate have been disclosed in the following U.S. patent applications: U.S. Ser. No. 10/853,585 to DeJovin, et al.; U.S. Ser. No. 10/626,037 to Scherer; U.S. Ser. No. 10/607,439 to Gil, et al.; and U.S. Ser. No. 10/763,807 to Shanler, et al.

### SUMMARY OF THE INVENTION

In one aspect, the present invention relates to pharmaceutical compositions for treating erythema associated with rosacea. The pharmaceutical compositions comprise brimonidine tartrate in an amount from about 0.17 percent by weight to about 0.19 percent by weight in a pharmaceutically acceptable carrier such as a gel or a cream. The amount of brimonidine tartrate in the composition is preferably from about 0.175 percent by weight to about 0.185 percent by weight, more preferably the brimonidine tartrate is present in the amount of 0.18 percent by weight.

In a preferred embodiment, the pharmaceutically acceptable carrier is a gel. The gel may include one or more skin-penetrating agents, moisturizers, preservatives, gelling agents, and protective agents.

The skin-penetrating agent may be present in an amount from about 2 percent by weight to about 10 percent by weight. The preferred skin-penetrating agent is propylene glycol.

The moisturizer is preferably present in an amount from about 2 percent by weight to about 10 percent by weight. The preferred moisturizer is glycerin.

The preservative may be present in an amount from about 0.1 percent by weight to about 1 percent by weight. The preferred preservatives are methylparaben and phenoxyethanol.

The gelling agent is preferably present in an amount from about 0.5 percent by weight to about 2 percent by weight. The preferred gelling agent is Carbomer 934P.

The protective agent may be present in an amount from about 0.1 percent by weight to about 1.5 percent by weight. The preferred protective agent is titanium dioxide.

Additionally, the composition may contain a sufficient amount of base to cause the carrier to have a pH of about 5 to about 7.5 when the gel is diluted by a factor of ten. Preferably, the pH range is about 6.2 to about 6.8 when the gel is diluted by a factor of ten. The preferred base is sodium or potassium hydroxide.

In a preferred embodiment, the composition includes water, a carbomer, propylene glycol, glycerin, methylparaben, phenoxyethanol, glycerin, titanium dioxide and a sufficient amount of base to cause the carrier to have a pH from about 6.2 to about 6.8 when the gel is diluted by a factor of ten.

The invention also relates to a method for treating erythema in patient with rosacea by topically administering brimonidine tartrate in an amount from about 0.17 percent by weight to about 0.19 percent by weight in a pharmaceutically acceptable cream or gel to the site of the erythema on the skin of the patient.

In a second aspect, the invention relates to methods of treating erythema in a patient with rosacea including topically administering brimonidine tartrate in an amount from about 0.17 percent by weight to about 0.19 percent by weight in a pharmaceutically acceptable cream or gel to the site of erythema on the skin of the patient. Preferably, the brimonidine tartrate is present in an amount from about 0.175 percent by weight to about 0.185 percent by weight. Most preferably, the brimonidine tartrate is present in an amount of about 0.18 percent by weight.

The carrier is preferably a gel or a cream. If the carrier is a gel, the gel preferably contains a skin-penetrating agent. The preferred skin-penetrating agent is propylene glycol. The gel may also contain a moisturizer. The preferred moisturizer is glycerin. The gel may also contain a preservative. The preferred preservatives include methylparaben and phenoxyethanol.

In a preferred embodiment, the gel contains a sufficient amount of base to cause the carrier to have a minimum pH of about 5 and a maximum pH of about 7.5 when the gel is diluted by a factor of ten. In another preferred embodiment, the gel contains a sufficient amount of base to cause the carrier to have a minimum pH of about 6.2 and a maximum pH of about 6.8. The preferred bases are sodium and potassium hydroxide.

The gel may contain a gelling agent. The preferred gelling gent is a carbomer. The gel may also contain a protective agent, a cosmetic agent, or a combination thereof. A preferred cosmetic agent is titanium dioxide.

In a preferred embodiment, the gel contains water, a gelling agent, a skin-penetrating agent, a moisturizer, a preservative, and a cosmetic agent. In another preferred embodiment, the gel comprises water, a carbomer, propylene glycol, glycerin, methylparaben, phenoxyethanol, glycerin, titanium dioxide and a sufficient amount of base to cause the carrier to have a minimum pH of about 6.2 and a maximum pH of about 6.8 when the gel is diluted by a factor of ten. The preferred base is sodium or potassium hydroxide.

### DESCRIPTION OF THE FIGURES

FIG. 1 shows the three-day average change in baseline CEA for all three visits over an eight hour period.

FIG. 2 shows the CEA does response, i.e., the change from the pre-dosage score, for each of the three dosage levels and the vehicle.

FIG. 3 shows the success rate. Patients were evaluated on day 28. Success was defined when a patient achieved a CEA score of 0 or 1, or a patient's erythema decreased by at least two points. The Y-axis, i.e., “% responder” is the percent of patients who achieved success over an eight hour period.

### DETAILED DESCRIPTION OF THE INVENTION

The present invention relates to an improved pharmaceutical composition comprising brimonidine tartrate in a pharmaceutically acceptable carrier such as a cream or gel. Brimonidine tartrate is effective in treating the symptoms of rosacea. Rosacea is an inflammatory skin disorder that generally affects the cheeks, nose, chin, and forehead of a patient. The major symptom of rosacea is erythema, i.e., the abnormal redness of the skin. The pharmaceutically acceptable composition of the present invention can be applied topically to the site of erythema on the skin of a patient.

It has unexpectedly been discovered that a composition having a narrow range of concentration of brimonidine tartrate has superior clinical properties, e.g., balance of efficacy and acceptable side effects.

Brimonidine tartrate, i.e., 5-bromo-6-(2-imidazolidinylideneamino)quinoxaline L-tartrate, is a selective alpha-2 adrenergic agonist. Its structure is shown below.

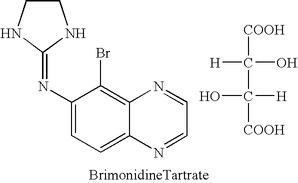

The pharmaceutical compositions of the invention contain brimonidine tartrate in an amount from about 0.17% by weight to about 0.19% by weight based upon the total weight of the composition. Preferably, the brimonidine tartrate is administered in an amount from about 0.175% by weight to about 0.185% by weight. Most preferably, the brimonidine tartrate is administered in an amount of about 0.18 percent by weight.

### Pharmaceutically Acceptable Carriers

In one embodiment, the pharmaceutically acceptable carrier is a gel. Gels are semisolid systems that contain suspensions of inorganic particles, usually small inorganic particles, or organic molecules, usually large organic molecules, interpenetrated by a liquid. When the gel mass comprises a network of small discrete inorganic particles, it is classified as a two-phase gel. Single-phase gels consist of organic macromolecules distributed uniformly throughout a liquid such that no apparent boundaries exist between the dispersed macromolecules and the liquid. Suitable gels for use in the invention are known in the art, and may be two-phase or single-phase systems. Some examples of suitable gels are disclosed in REMINGTON: THE SCIENCE AND PRACTICE OF PHARMACY 1517-1518 (Alfonso R. Gennaro ed. 19^{th} ed. 1995). Other suitable gels for use with the invention are disclosed in U.S. Pat. No. 6,387,383 (issued May 14, 2002); U.S. Pat. No. 6,517,847 (issued Feb. 11, 2003); and U.S. Pat. No. 6,468,989 (issued Oct. 22, 2002).

Gelling agents, that may be used include those known to one skilled in the art, such as hydrophilic and hydroalcoholic gelling agents frequently used in the cosmetic and pharmaceutical industries. Preferably, the hydrophilic or hydroalcoholic gelling agent comprises “CARBOPOL®” (B.F. Goodrich, Cleveland, Ohio), “HYPAN®” (Kingston Technologies, Dayton, N.J.), “NATROSOL®” (Aqualon, Wilmington, Del.), “KLUCEL®” (Aqualon, Wilmington, Del.), or “STABILEZE®” (ISP Technologies, Wayne, N.J.).

“CARBOPOL®” is one of numerous cross-linked acrylic acid polymers that are given the general adopted name carbomer. “Carbomer” is the USP designation for various polymeric acids that are dispersible but insoluble in water. When the acid dispersion is neutralized with a base a clear, stable gel is formed. The preferred carbomer is Carbomer 934P because it is physiologically inert and is not a primary irritant or sensitizer. Other carbomers include 910, 940, 941, and 1342.

Carbomers dissolve in water and form a clear or slightly hazy gel upon neutralization with a caustic material such as sodium hydroxide, potassium hydroxide, triethanolamine, or other amine bases. “KLUCEL®.” is a cellulose polymer that is dispersed in water and forms a uniform gel upon complete hydration. Other preferred gelling agents include hydroxyethylcellulose, cellulose gum, MVE/MA decadiene crosspolymer, PVM/MA copolymer, or a combination thereof.

In a preferred embodiment, the minimum amount of gelling agent in the composition is about 0.5%, more preferably, about 0.75%, and most preferably about 1%.

In another preferred embodiment, the maximum amount of gelling agent in the composition is about 2%, more preferably about 1.75%, and most preferably about 1.5%.

The pharmaceutical carrier may also be a cream. A cream is an emulsion, i.e., a dispersed system comprising at least two immiscible phases, one phase dispersed in the other as droplets ranging in diameter from 0.1 μm to 100 μm. An emulsifying agent is typically included to improve stability. When water is the dispersed phase and an oil is the dispersion medium, the emulsion is termed a water-in-oil emulsion. When an oil is dispersed as droplets throughout the aqueous phase as droplets, the emulsion is termed an oil-in-water emulsion. Emulsions that can be used as topical carriers and their preparation are disclosed in REMINGTON: THE SCIENCE AND PRACTICE OF PHARMACY 282-291 (Alfonso R. Gennaro ed. 19^{th} ed. 1995).

The pH of the pharmaceutical carrier is adjusted with, for example, a base such as sodium hydroxide or potassium hydroxide. The minimum pH of the carrier is about 5, preferably 5.5, and most preferably 6.2 when the carrier is diluted by a factor of ten. The maximum pH of the carrier is about 7.5, preferably 7, and most preferably 6.8 when the carrier is diluted by a factor of ten. Each minimum pH value can be combined with each maximum pH value to create various pH ranges. For example, the pH may be a minimum of 6.2 and a maximum of 7.5.

The pH values given above are those that occur if the composition is diluted with water by a factor of ten. It is not necessary to dilute the composition by a factor of ten in order to obtain a pH value. In practice, the composition may be diluted by any value that permits pH to be measured. For example, the composition may be diluted by a factor of about five to about twenty.

### Excipients

The compositions of the invention may include pharmaceutically acceptable excipients including, but not limited to, protective agents, adsorbents, preservatives, moisturizers, and skin-penetration agents. Pharmaceutically acceptable excipients are listed in REMINGTON: THE SCIENCE AND PRACTICE OF PHARMACY 866-885(Alfonso R. Gennaro ed. 19^{th} ed. 1995; Ghosh, T. K.; et al. TRANSDERMAL AND TOPICAL DRUG DELIVERY SYSTEMS (1997).

Suitable protective agents and/or cosmetic agents, and adsorbents include, but are not limited to, dusting powders, zinc sterate, collodion, dimethicone, silicones, zinc carbonate, aloe vera gel and other aloe products, vitamin E oil, allatoin, petrolatum, titanium dioxide, and zinc oxide. The preferred protective agent is titanium dioxide.

In a preferred embodiment, the minimum amount of cosmetic agent in the composition is about 0.01%, more preferably, about 0.025%, and most preferably about 0.05%.

In another preferred embodiment, the maximum amount of cosmetic agent in the composition is about 0.15%, more preferably about 0.1%, and most preferably about 0.075%.

Suitable preservatives include, but are not limited to, quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol; parabens such as methylparaben, ethylparaben, propylparaben, and butylparaben; antibacterial esters, for example, esters of parahydroxybenzoic acid; and other anti-microbial agents such as chlorhexidine, chlorocresol, benzoic acid, polymyxin, and phenoxyethanol. The preferred preservatives are methylparaben and phenoxyethanol.

In a preferred embodiment, the minimum amount of preservative in the composition is about 0.1%, more preferably, about 0.2%, and most preferably about 0.3%.

In another preferred embodiment, the maximum amount of preservative in the composition is about 1%, more preferably about 0.75%, and most preferably about 0.5%.

Suitable moisturizers include, but are not limited to, glycerin, sorbitol, polyethylene glycols, urea, and propylene glycol. The preferred moisturizer is glycerin.

In a preferred embodiment, the minimum amount of moisturizer in the composition is about 2%, more preferably, about 3.5%, and most preferably about 4.5%.

In another preferred embodiment, the maximum amount of moisturizer in the composition is about 10%, more preferably about 8%, and most preferably about 6%.

Suitable skin-penetration agents include, but are not limited to, ethyl alcohol, isopropyl alcohol, octylphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate); and N-methylpyrrolidone. The preferred skin-penetrating agent is propylene glycol.

In a preferred embodiment, the minimum amount of skin-penetrating agent in the composition is about 2%, more preferably, about 3.5%, and most preferably about 4.5%.

In another preferred embodiment, the maximum amount of skin-penetrating agent in the composition is about 10%, more preferably about 8%, and most preferably about 6%.

### Topical Administration

In a preferred embodiment, the pharmaceutical composition is delivered topically to the affected area of the skin. To treat the symptoms of rosacea and more specifically erythema, the pharmaceutical compositions of the invention are topically applied directly to the affected area in any conventional manner well known in the art. For example, the compositions are applied by cotton swab or applicator stick, or by simply spreading a formulation of the invention onto the affected area with fingers. Generally the amount of a topical formulation of the invention applied to the affected skin area ranges from about 0.0001 g/cm² of skin surface area to about 0.01 g/cm², preferably, 0.001 g/cm² to about 0.003 g/cm² of skin surface area. Typically, one to four applications per day are recommended during the term of treatment.

### EXAMPLES

### Example 1

### Synthesis of Brimonidine (5-Bromoquinoxalin-6-yl)-(4,5-dihydro-1H-imidazol-2-yl)-amine

To a stirred solution of 6-amino-5-bromoquinoxaline hydrobromide (10 g) in distilled water (150 ml) is added thiophosgene (3 ml). The solution is stirred for two hours at room temperature and the resultant precipitate is collected by filtration, washed with water, and dried to afford 5-bromo-6-isothiocyanato-quinoxaline.

The 5-bromo-6-isothiocyanato-quinoxaline (3.5 g) is directly dissolved in benzene (400 ml) and added dropwise to a well-stirred solution of ethylene diamine (15 g.) in benzene (50 ml). During a period of about two hours, an oil separates as a lower layer. The upper benzene layer is poured off and the oil is washed with diethyl ether and then dissolved in methanol (500 ml). The methanolic solution is refluxed until hydrogen sulfide evolution ceases. The methanolic solution is concentrated in vacuo to a volume of approximately 100 ml upon which a yellow solid precipitates. The precipitate is collected by filtration and recrystallized from methanol to afford of (5-Bromo-quinoxalin-6-yl)-(4,5-dihydro-1H-imidazol-2-yl)-amine: m.p. 250-251° C.

### Example 2

### Synthesis of Brimonidine Tartrate 5-bromo-6-(2-imidazolidinylideneamino)quinoxaline L-tartrate

The tartrate salt of brimonidine can be synthesized by adding (L)-(+)-tartaric acid to a solution of brimonidine in aqueous methanol. The brimonidine tartrate will separate out of solution.

### Example 3

Gel Formulation

| Ingredient | Weight Percent |
|---|---|
| Brimonidine tartrate | 0.18% |
| Carbomer 934P | 1.25% |
| Methylparaben | 0.3% |
| Phenoxyethanol | 0.4% |
| Glycerin | 5.5% |
| 10% Titanium dioxide | 0.625% |
| Propylene glycol | 5.5% |
| 10% NaOH Solution | 6.5% |
| DI Water | QS |
| TOTAL | 100% |

### Example 4

Cream Formulation

| Ingredient | Weight Percent |
|---|---|
| Brimonidine tartrate | 0.18% |
| Phenoxyethanol | 0.8% |
| Methylparaben | 0.2% |
| Propylparaben | 0.05% |
| Disodium EDTA | 0.01% |
| Butylated Hydroxytoluene | 0.05% |
| PEG-300 | 4.0% |
| PEG-6 Stearate (and) | 7.5% |
| Glycol Stearate (and) PEG- | |
| 32 Stearate | |
| Cetostearyl alcohol | 4.0% |
| Caprylic capric | 7.0% |
| triglycerides | |
| Diisopropyl adipate | 7.0% |
| Oleyl alcohol | 7.0% |
| Lanolin USP | 2.0% |
| Ceteareth-6 (and) Stearyl | 2.0% |
| Alcohol | |
| Ceteareth-25 | 2.0% |
| Tartaric Acid | 0.001% |
| DI Water | 56.209% |
| TOTAL | 100% |

### Example 5

### Clinical Study of Brimonidine Tartrate Gels

A double-blind, placebo-controlled study was conducted at six centers of 110 patients with moderate to severe erythema. Patients were administered a gel similar to the formulation in Example 3, which contained either a “low” dosage of brimonidine tartrate (0.02% by weight), a “mid” dosage of brimonidine tartrate (0.07% by weight), a “high” dosage of brimonidine tartrate (0.20% by weight), or no brimonidine tartrate (“vehicle” or placebo group). (These concentrations are greater or less than the claimed ones.) The treatment lasted for 28 days during which time patients applied the gel each day. On days 1, 14, and 28, patients applied the gel under the supervision of clinical staff in the study centers, and were evaluated at set intervals for up to eight hours.

Evaluation led to a Clinician's Erythema Assessment Score (CEA). Each patients' erythema was rated on a scale from 0 to 4, with 0 being the rating for no erythema and 4 being the rating for extreme erythema. Each patient selected for this study initially had a score of 3 or 4.

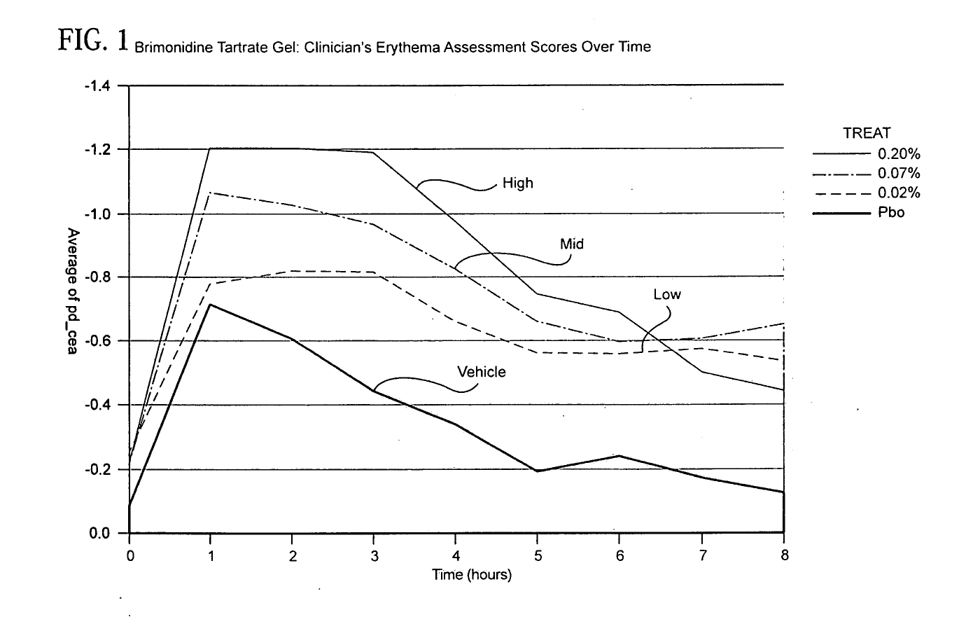

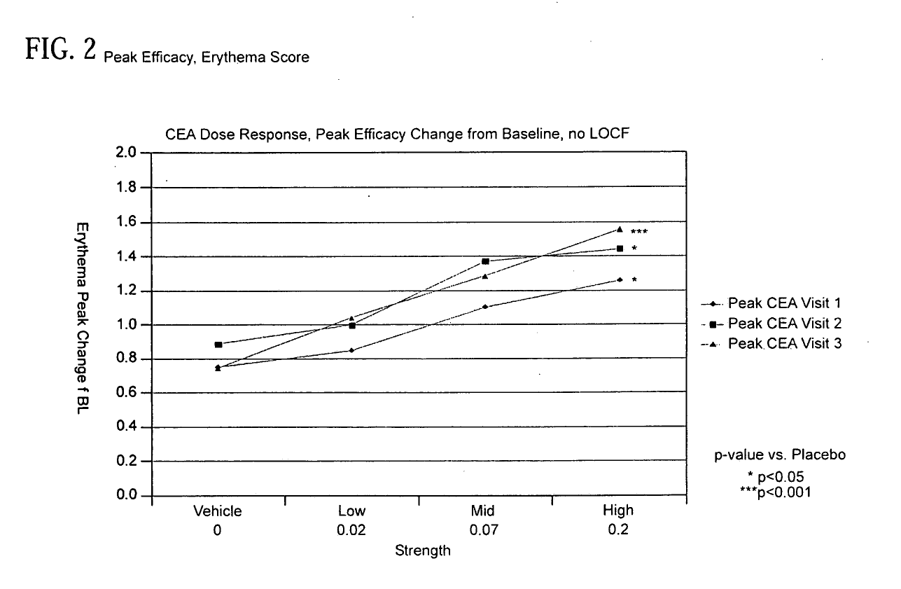

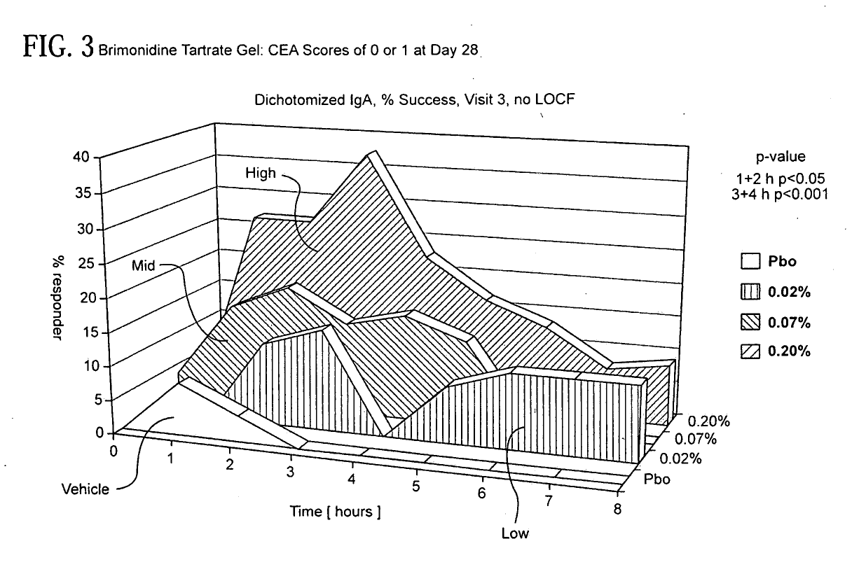

A statistically significant reduction in the average CEA was seen across all visits in the high dosage group (p<0.001). Also, a side effect profile was established. Results are shown in FIGS. 1-3.

We claim:

1. A pharmaceutical composition comprising brimonidine tartrate in an amount from about 0.17 percent by weight to about 0.19 percent by weight in a pharmaceutically acceptable carrier selected from the group consisting of a gel and a cream.

2. A composition according to claim 1, wherein the brimonidine tartrate is present in an amount from about 0.175 percent by weight to about 0.185 percent by weight.

3. A composition according to claim 1, wherein the brimonidine tartrate is present in an amount of about 0.18 percent by weight.

4. A composition according to claim 1, wherein the carrier is a gel.

5. A composition according to claim 1, wherein the carrier is a cream.

6. A composition according to claim 4, wherein the gel comprises a skin-penetrating agent.

7. A composition according to claim 6, wherein the skin-penetrating agent is propylene glycol.

8. A composition according to claim 4, wherein the gel comprises a moisturizer.

9. A composition according to claim 8, wherein the moisturizer is glycerin.

10. A composition according to claim 4, wherein the gel comprises a preservative.

11. A composition according to claim 10, wherein the preservative is methylparaben.

12. A composition according to claim 10, wherein the preservative is phenoxyethanol.

13. A composition according to claim 4, wherein the gel comprises a sufficient amount of base to cause the carrier to have a minimum pH of about 5 and a maximum pH of about 7.5 when the gel is diluted by a factor of ten.

14. A composition according to claim 13, wherein the pH has a minimum of about 6.2 and a maximum of about 6.8 when the gel is diluted by a factor of ten.

15. A composition according to claim 13, wherein the base is sodium or potassium hydroxide.

16. A composition according to claim 4, wherein the gel comprises a gelling agent.

17. A composition according to claim 16, wherein the gelling agent is a carbomer.

18. A composition according to claim 4, wherein the gel comprises a protective agent, a cosmetic agent, or a combination thereof.

19. A composition according to claim 18, wherein the cosmetic agent is titanium dioxide.

20. A composition according to 4, wherein the gel comprises water, a gelling agent, a skin-penetrating agent, a moisturizer, a preservative, and a cosmetic agent.

21. A composition according to claim 20, wherein the gel comprises water, a carbomer, propylene glycol, glycerin, methylparaben, phenoxyethanol, glycerin, titanium dioxide and a sufficient amount of base to cause the carrier to have a minimum pH of about 6.2 and a maximum pH of about 6.8 when the gel is diluted by a factor of ten.

22. A composition according to claim 21, wherein the base is sodium or potassium hydroxide.

23. A method for treating erythema in a patient with rosacea, the method comprising topically administering brimonidine tartrate in an amount from about 0.17 percent by weight to about 0.19 percent by weight in a pharmaceutically acceptable cream or gel to the site of erythema on the skin of the patient.
